(19) United States
(12) Patent Application Publication
(10) Pub. No.: US 2021/0260071 A1
(43) Pub. Date: Aug. 26, 2021

(54) AQUEOUS SOLUTION FOR A SURGERY AND METHOD FOR USING THE SAME

(71) Applicant: TAMER AHMET SECKIN, NEW YORK, NY (US)

(72) Inventor: TAMER AHMET SECKIN, NEW YORK, NY (US)

(21) Appl. No.: 16/798,371

(22) Filed: Feb. 23, 2020

(51) Int. Cl. A61K 31/5415; A61K 31/375; A61K 31/355; A61K 31/07; A61K 38/095; A61K 31/167; A61B 17/3205; A61B 1/313; A61M 3/02

(52) U.S. Cl. CPC A61K 31/5415; A61K 31/375; A61K 31/355; A61K 31/07; A61B 2017/00818; A61K 31/167; A61B 17/3205; A61B 1/3132; A61M 3/0254; A61K 38/095

(57) ABSTRACT

The present invention provides an aqueous solution for a surgery, especially laparoscopic surgery and a method for using such a liquid solution during surgery. The aqueous solution comprises at least an antioxidant agent and a haemostatic agent in order to prevent intestinal obstruction, pain and difficulties in repeated surgeries. The present invention provides also a method for using such an aqueous solution during a surgery.

### TECHNICAL FIELD

The present invention relates to an aqueous solution for controlling oxidative stress and enhanced visual recognition during a surgery, especially laparoscopic surgery and a method for using such an aqueous solution during a surgery.

### BACKGROUND ART

Endometriosis, a disease affecting approximately 10% of the female population, requires laparoscopy and histological confirmation for diagnosis. Laparoscopic recognition of endometriosis lesions can be extremely challenging even for the experienced surgeon due to the protean appearances of endometriosis. Visual appearance of what is classically described as pigmented, dark lesions are easily spotted while the non-pigmented, more prevalent white lesions, also known as “subtle” lesions, pose difficulty for recognition. This is due to a wide unrestricted light spectrum, light reflection, and gas pressure used in laparoscopic surgery. An endometriosis surgeon should be familiar with all appearances of endometriosis. The classical or typical lesions are quickly visualized with varying colors from red to black, but they are always outnumbered by atypical and microscopic endometriosis, which is not easily recognized. The inexperienced surgeons who do not practice excision technique may miss these occult and deep lesions. A wide variety of angiogenesis and inflammatory process is taking place in the peritoneum and must be recognized in addition to the typical and atypical lesions of endometriosis.

US patent application US20130131457A1, which is incorporated here by reference, describes the use of a liquid for pressurizing the pelvic cavity instead of a gas. Such a liquid may typically be a saline solution such as normal saline (0.90% w/v of NaCl, about 300 mOsm/L or 9.0 g per liter).

Endoscopes having Near Band Imaging (NBI) technology are developed that enhance microvasculature and surface morphology. However, there are factors which are not under the control e.g. when users mix components from various manufacturers or when users adjust the controls on the monitor to their own liking. Further, the level of tissue illumination is often very uneven due to the nature of endoscopy and color varies with brightness, whereby brightness varies inversely by the square of the distance. Therefore, even if an endoscope provides accurate color reproduction under ideal test conditions, actual tissue color will vary constantly during live endoscopy.

US patent application US20160199514A1, which is incorporated here by reference, describes a liquid composition comprising a contrast enhancing agent for laparoscopic surgery in order to enhance the view of the surgeon during a laparoscopy, especially for detection of superficial collateral pathology, Leopard Spots, retroperitoneal fibrosis and micro endometriosis implants. Changing the color spectrum and using hydrofloatation with contrast color and retroperitoneal distention helps to visualize endometriosis otherwise undetectable by standard laparoscopic inspection. Further, the patent application US20160199514A1 discloses a method comprising introducing at least 100 mL liquid solution comprising a contrast enhancing agent into the retroperitoneal space through a catheter or a channel of a laparoscope using an irrigation pump for hydro-floatation in the retroperitoneal space and hydro-distention of peritoneum to create contrast under peritoneal membrane. The patent application US20160199514A1 discloses distending the peritoneum and trapping the colored solution comprising the contrast enhancing agent in the fibrillary collagen based aerolar tissue among blood vessels, nerves and organs. The patent application US20160199514A1 discloses distending the peritoneum such that the pressure in the abdominal space or in the retroperitoneal space is arranged as less than the venous pressure of the patient, specifically up to 80 mmHg or calibrated according to the venous pressure of the patient. The contrast enhancing agent used in the solution is one of Methylene Blue (having the formula C₁₆H₁₈ClN₃S and designated as CI 52015; C₁₆H₁₈N₃SCl); 3,7-bis(dimethylamino)-phenothiazin-5-ium chloride), Indigo Carmine, Indocyanine Green and any combination of Methylene Blue, Indigo Carmine or Indocyanine Green.

The solution disclosed by the patent application US20160199514A1 makes use of Methylene Blue essentially as a dye and does not consider therapeutic effects on the excision site. Recent studies have put the attention on the role of oxidative stress, defined as an imbalance between reactive oxygen species (ROS) and antioxidants, which may be implicated in the pathophysiology of endometriosis causing a general inflammatory response in the peritoneal cavity which may lead to pain and peritoneal adhesions. Further, bleeding during surgery, blood clots and fibrin deposits induce adhesions. Therefore, each surgery causes patient more adhesions of peritoneum and intra peritoneal organs. These adhesions may cause intestinal obstruction, pain and difficulties in repeated surgeries.

The haemostatic control of bleeding is very important for prevention of adhesions caused by blood clots and fibrin deposits. Conventional methods of administration of haemostatic agents in therapeutic doses frequently is associated with significant adverse side effects. Generally, vasopressin is administered by injection to the excision site. However, injecting vasopressin to the patient may cause intraoperative cardiovascular complications. Especially, during a surgery with a plurality of excision sites, i.e. due to distributed endometrioma, controlling the dosage of injected vasopressin may be difficult.

There is a need for prevention of intestinal obstruction, pain and difficulties in repeated surgeries. Further, there is a need for prevention of adhesions due to a laparoscopic surgery while administering haemostatic agents in optimum dosage. There is also a need for prevention of recurring of endometriosis after a laparoscopic surgery while controlling oxidative stress in the peritoneum without adding further steps to the surgical procedure.

### DISCLOSURE OF THE PRESENT INVENTION

The present invention provides an aqueous solution for introducing into a site of surgery or into an abdominal cavity during laparoscopic surgery comprising at least an antioxidant agent and a haemostatic agent. The aqueous solution may comprise as solvent: sterile water, Normal Saline, or an isotonic solution containing 6.5 g NaCl, 0.42 g KCl, 0.25 g CaCl₂ and 0.2 g of sodium bicarbonate dissolved in one liter of distilled water. The aqueous solution may comprise at least 100 mL, preferably 100 mL to 3 L, more preferably 1 L or 3 L solvent. The antioxidant agent may comprise Methylene Blue dye. The aqueous solution may comprise 1 to 3% or about 1% Methylene Blue. Present invention makes use of Methylene Blue as a dye and as a medication. A further antioxidant agent may be Vitamin C or one of the Vitamins C, E and A or a combination thereof. The aqueous solution may comprise about 500 mg/3000 cc Vitamin C. The haemostatic agent may comprise a local vasoconstructive agent or Vasopressin. The aqueous solution may comprise about 0.01% Vasopressin. The aqueous solution may further comprise a local anesthetic. The local anesthetic may comprise about 1% Lidocain.

The aqueous solution of the present invention enhances the view during the surgery for naked eyes of a surgeon by distending and floating the site of surgery while mitigating at the same time bleeding due to surgery and controlling oxidative stress by acting on the site of surgery.

The aqueous solution of the present invention provides a contrast enhanced view of the peritoneum during a laparoscopy while keeping the peritoneum distended due to applied pressure and prevents adhesion formation, oxidative stress and excessive bleeding.

In a further aspect, the present invention provides a method for administering an aqueous solution into an abdominal cavity during laparoscopic surgery comprising distending the peritoneum by introducing the aqueous solution and trapping the aqueous solution in the peritoneum, wherein the aqueous solution comprises an antioxidant and a haemostatic agent dissolved in at least 100 mL, preferably 100 mL to 3 L, more preferably 1 L or 3 L solvent.

The additives in the aqueous solution of the present invention prevent adhesion formation, oxidative stress and excessive bleeding by acting on the surfaces of all excised areas forming a preventive and protective barrier postoperatively. Further, retroperitoneal hydro-distention makes it possible to identify peritoneal micro defects to determine excision boundaries and inspection of retroperitoneal space. Further, creation of blue color contrast in the background makes it possible to identify peritoneal micro defects to determine excision boundaries and inspection of retroperitoneal space under blue colored solution for micro invasion and fibrosis.

The solution of the present invention may be used in all types of surgeries including all abdominal and thoracic surgeries on behalf of its antioxidant, adhesion preventive and bleeding control manner.

The technique of the present invention easily identifies normal texture of the peritoneum. This method assists the surgeon to target the lesion by clear recognition of subtle peritoneal changes in endometriosis. Assisted by enhancing contrast and eliminating the yellow and red spectrums, the surgeon is more easily able to perform tedious and precisely accurate excision surgery without unnecessary removal of normal peritoneum. Changing the color spectrum and using hydrofloatation with contrast color and retroperitoneal distention helps to visualize endometriosis otherwise undetectable by standard laparoscopic inspection.

The method according to the present invention comprises introducing the solution and the contrast enhancing agent into the abdominal cavity, preferably into the retroperitoneal space, preferably through a catheter or a channel of a laparoscope, preferably using an irrigation pump. Preferably 100 mL to 3 L, more preferably 100 mL or 3 L, of the solution may be introduced for hydro-floatation in the retroperitoneal space and hydro-distention of peritoneum. Accordingly, the method according to the present invention comprises distending the peritoneum and trapping the solution in the peritoneum.

According to the invention, in order to provide a colored solution, 1 to 250 mg, preferably 10 to 50 mg, more preferably 30 mg contrast enhancing agent may be dissolved in 1 L sterile water or preferably in Normal Saline, more preferably in an isotonic solution containing 6.5 g NaCl, 0.42 g KCl, 0.25 g CaCl₂ and 0.2 g of sodium bicarbonate dissolved in one liter of distilled water, more preferably containing per 1000 mL:
130 to 131 mEq of sodium ion=130-131 mmol/L
109 to 111 mEq of chloride ion=109-111 mmol/L
28 to 29 mEq of lactate=28-29 mmol/L
4 to 5 mEq of potassium ion=4-5 mmol/L
3 to 4 mEq of calcium ion=1.5-2 mmol/L
   more preferably containing per 1000 mL:
130 mEq of sodium ion=130 mmol/L
109 mEq of chloride ion=109 mmol/L
28 mEq of lactate=28 mmol/L
4 mEq of potassium ion=4 mmol/L
3 mEq of calcium ion=15 mmol/L
   or:
131 mEq of sodium ion=131 mmol/L
111 mEq of chloride ion=111 mmol/L
29 mEq of lactate=29 mmol/L
5 mEq of potassium ion=5 mmol/L
4 mEq of calcium ion=2 mmol/L.

Each 100 mL of such an isotonic solution may contain: Sodium chloride 600 mg; sodium lactate, anhydrous 310 mg; potassium chloride 30 mg; calcium chloride, dihydrate 20 mg. Such an isotonic solution may further contain sodium hydroxide and may further contain hydrochloric acid for pH adjustment. Heparin may be added to such a solution (5 IU per L).

Calcium Chloride, USP is chemically designated calcium chloride, dihydrate (CaCl₂⋅₂H₂O), white fragments or granules freely soluble in water. Potassium Chloride, USP is chemically designated KCI, a white granular powder freely soluble in water. Sodium Chloride, USP is chemically designated NaCl, a white crystalline powder freely soluble in water. Sodium Lactate, USP is chemically designated NaC₃H₅O₃, a 60% aqueous solution miscible in water.

During electro-surgical procedures, instead of an electrolyte solution, % 1.5 Glycine (NH2CH2COOH) solution 1.5 g glycine per 100 mL water solution may be used in a method according to the invention. Alternatively, a 5% Mannitol solution (5 g Mannitol per 100 mL) or a solution containing Sorbitol 2.70 g and Mannitol 0.54 g in 100 mL sterile water. Mannitol, USP is chemically designated D-mannitol (C6H14O6), white crystalline powder or free-flowing granules, freely soluble in water. Sorbitol, NF is chemically designated D-glucitol (C6H14O6), white powder, granules or flakes very soluble in water.

Alternatively, 4% icodextrin solution may be used as a solution during the method according to the invention.

Present invention provides a method for using an aqueous solution comprising introducing the aqueous solution into a peritoneum of a patient during a laparoscopic surgery to float the tissues in the peritoneum, viewing and locating a site for excision under the aqueous solution, excision of the site under the aqueous solution. The method may further comprises trapping the aqueous solution in the peritoneum to distend the peritoneum; introducing at least 100 mL, preferably 100 mL to 3 L, more preferably 1 L or 3 L of the aqueous solution into peritoneum; further adding of the aqueous solution onto the site of excision before completing the surgery to cover the site of excision with the aqueous solution; leaving some of the aqueous solution within peritoneum enough to cover the site of excision.

These and other objects of the present invention will be apparent from the drawings, claims and detailed descriptions herein.

### BRIEF DESCRIPTION OF THE DRAWINGS

The accompanying drawings, referred to herein and constituting a part hereof, illustrate preferred embodiments of the present invention and together with the description, serve to explain the principles of the present invention, wherein:

FIG. 1 is a partial cross-sectional side view of a female body comprising an embodiment of the present invention depicting a procedure of a laparoscopic surgery;

FIG. 2 is a partial cross-sectional side view of a female body comprising an embodiment of the present invention depicting the procedure after the laparoscopic surgery;

FIG. 3 shows the abdominal cavity of FIG. 1 in a perspective view.

### DESCRIPTION OF AN EXEMPLARY EMBODIMENT

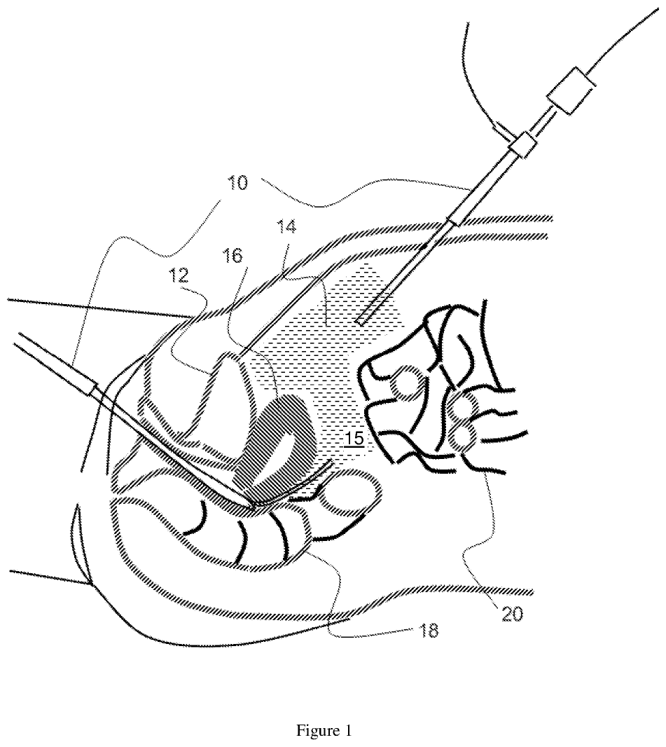

Referring to FIG. 1, a partial female body is illustrated in cross-sectional view showing the peritoneum (15), laparoscopes (10) placed through the abdominal wall and the transvaginal-transdouglas route, the uterus (16), the bladder (12), the rectum (18) and the small intestine (20).

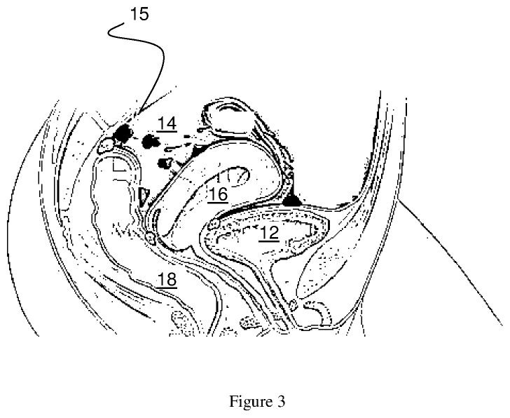

According to an embodiment of the present invention shown in FIG. 1, about 1 L of an aqueous solution (14) of the present invention has been introduced into the peritoneum (15) comprising 500 mg/3000 cc Vitamin C, 0.01% Vasopressin, 1% Lidocain and 1% Methylene Blue dye. However another composition may be introduced as described throughout this specification. The blue colored solution (14) distends the peritoneum, makes the tissues in the retroperitoneum (15) float and provides contrast in the background of the distended peritoneum (15) and in the retroperitoneum (15) during near contact scanning with the laparoscope (10). Although a laparoscopy has been illustrated by this embodiment, the aqueous solution of the present invention may also be used in a laparotomy. FIG. 3 shows the surgery site of FIG. 1 in a different perspective. The blue colored solution (14) provided here in the retroperitoneum (15) background contrast for recognizing subtle endometriosis around the uterus (16) and the bladder (12).

The aqueous solution (14) of this embodiment comprises Methylene Blue and Vitamin C as antioxidant agents and Vasopressin as haemostatic agent. The aqueous solution (14) comprises Normal Saline as isotonic solution. The aqueous solution (14) comprises further Lidocain as a local anesthetic.

According to this embodiment, the inventive method comprises introducing the aqueous solution (14) into peritoneum (15) of a patient during a laparoscopic endometriosis surgery to float the tissues in the peritoneum (15), viewing and locating a tissue having endometriosis for excision under the aqueous solution (14), and excision of the tissue having endometriosis under said aqueous solution (14). According to this embodiment, the aqueous solution (14) has been trapped in the peritoneum (15) to distend the abdominal cavity. 1 L of said aqueous solution was introduced into peritoneum (15). According this embodiment, the method further comprises further addition of the aqueous solution (14) onto the site of excision and covering said site of excision with the aqueous solution (14) before completing the surgery, hence a part of the aqueous solution (14) is left within peritoneum (15) covering the site of excision. According this embodiment the aqueous solution (14) is filled into the abdominal cavity by an irrigation pump.

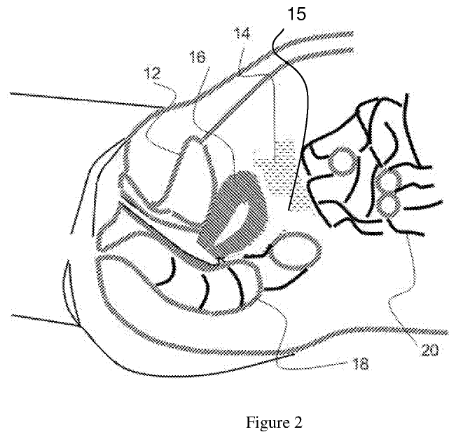

Referring to FIG. 2, the partial female body shown in FIG. 1 is illustrated in cross-sectional view showing the peritoneum (15), the uterus (16), the bladder (12), the rectum (18) and the small intestine (20) after the surgery procedure. As shown in FIG. 2, just the site of excision is covered with the inventive aqueous solution (14), since the inventive aqueous solution (14) was removed partially from peritoneum (15) by absorption into the body before completing the surgery and a part of the inventive aqueous solution (14) was left within the abdominal cavity enough to cover the site of excision and the pressure within the abdominal cavity has been reduced and the distension of the peritoneum has been resolved.

All abnormal peritoneum visually recognized by nearcontact laparoscopic inspection were excised using cold scissors. Retroperitoneum of the pelvic sidewalls were hydrodistended with Methylene Blue containing aqueous solution. Elimination of red, yellow and white hues allow background contrast for visual assistance recognizing nonpigmented foci of peritoneal thickening suggestive of subtle endometriosis. Elimination of high-end spectrum of light using the inventive solution with hydrodistention of the retroperitoneum enhances the surgeon's vision.

While the above description contains many specifics, these specifics should not be construed as limitations on the scope of the invention, but merely as exemplifications of preferred embodiments thereof. Those skilled in the art will envision many other possible variations that are within the scope and spirit of the invention as defined by the claims appended hereto.

1. Aqueous solution for controlling oxidative stress and enhanced visual recognition during a surgery, comprising Methylene Blue and a haemostatic agent.

2. Aqueous solution according to claim 1, comprising one of the following as solvent:
a. sterile water
b. Normal Saline, or
c. an isotonic solution containing about 6.5 g NaCl, 0.42 g KCl, 0.25 g CaCl₂ and 0.2 g of sodium bicarbonate dissolved in one liter of distilled water.

3. Aqueous solution according to claim 1, comprising at least 100 mL, preferably 100 mL to 3 L, more preferably 1 L or 3 L solvent.

4. Aqueous solution according to claim 1, comprising an antioxidant agent in addition to Methylene Blue dye.

5. Aqueous solution according to claim 1, comprising 1 to 3% or about 1% Methylene Blue or per 1000 mL, 1 to 250 mg of Methylene Blue or per 100 mL, 1 to 5 mg Methylene Blue, more preferably 3 mg Methylene Blue.

6. Aqueous solution according to claim 1, comprising an antioxidant agent in addition to Methylene Blue dye in form of one of the Vitamins C, E and A or a combination thereof.

7. Aqueous solution according to claim 1, comprising about 500 mg/3000 cc Vitamin C as an antioxidant agent in addition to Methylene Blue dye.

8. Aqueous solution according to claim 1, wherein said haemostatic agent comprises a local vasoconstructive agent or Vasopressin.

9. Aqueous solution according to claim 1, comprising about 0.01% Vasopressin.

10. Aqueous solution according to claim 1, further comprising a local anesthetic.

11. Aqueous solution according to claim 1, comprising about 1% Lidocain.

12. Method for using an aqueous solution according to claim 1 for controlling oxidative stress and enhanced visual recognition during a surgery, comprising:
a. introducing said aqueous solution into a site of surgery to float the tissues in said site of surgery;
b. viewing and locating a part of said site under said aqueous solution for excision; and
c. excision of said part under said aqueous solution.

13. Method according to claim 12, wherein said site of surgery is a peritoneum, an abdominal cavity or a thoracic site of a patient and said surgery comprises a laparoscopy and/or a laparotomy.

14. Method according to claim 12, further comprising trapping said aqueous solution in the peritoneum to distend the peritoneum.

15. Method according to claim 12, further comprising introducing at least 100 mL, preferably 100 mL to 3 L, more preferably 1 L or 3 L of said aqueous solution into abdominal cavity.

16. Method according to claim 12, further comprising further adding of said aqueous solution onto said site of excision before completing the surgery.

17. Method according to claim 12, further comprising covering said site of excision with said aqueous solution before completing the surgery.

18. Method according to claim 12, further comprising leaving some of said aqueous solution within the abdominal cavity to cover said site of excision.

19. Method according to claim 12, further comprising introducing said aqueous solution is performed by an irrigation pump.
